(19) United States
(12) Patent Application Publication
(10) Pub. No.: US 2010/0278749 A1
(43) Pub. Date: Nov. 4, 2010

(54) NANOPARTICLE CONTRAST AGENTS FOR DIAGNOSTIC IMAGING

(75) Inventors: Peter John Bonitatibus, JR., Saratoga Springs, NY (US); Matthew David Butts, Rexford, NY (US); Robert Edgar Colborn, Niskayuna, NY (US); Amit Mohan Kulkarni, Clifton Park, NY (US); Bruce Allan Hay, Niskayuna, NY (US); Andrew Soliz Torres, Troy, NY (US); Brian Christopher Bales, Niskayuna, NY (US); Michael Ernest Marino, Clifton Park, NY (US)

Correspondence Address: GENERAL ELECTRIC COMPANY;GLOBAL RESEARCH, ONE RESEARCH CIRCLE, BLDG. K1-3A59, NISKAYUNA, NY, 12309, US

(73) Assignee: GENERAL ELECTRIC COMPANY, SCHENECTADY, NY (US)

(21) Appl. No.: 12/431,899

(22) Filed: Apr. 29, 2009

(51) Int. Cl. A61K 49/04; A61K 49/18; G01V 3/00; G01N 23/04

(52) U.S. Cl. 424/9.32; 424/9.3; 424/9.4; 424/9.42; 324/307; 378/62; 977/773; 977/928; 977/930

(57) ABSTRACT

Compositions of nanoparticles functionalized with at least one net positively charged group and at least one net negatively charged group, methods for making a plurality of nanoparticles, and methods of their use as diagnostic agents are provided. The nanoparticles have characteristics that result in minimal retention of the particles in the body compared to other nanoparticles. The nanoparticle comprises a core and a shell. The shell comprises a plurality of silane moieties; at least one silane moiety of the plurality is functionalized with a net positively charged group and at least one silane moiety of the plurality is functionalized with a net negatively charged group.

### BACKGROUND

This application relates generally to contrast agents for diagnostic imaging, such as for use in X-ray/Computed Tomography (CT) or Magnetic Resonance Imaging (MRI). More particularly, the application relates to nanoparticle-based contrast agents, and methods for making and using such agents.

Almost all clinically approved diagnostic contrast agents are small molecule based. Iodinated aromatic compounds have served as standard X-ray or CT contrast agents, while Gd-chelates are used for Magnetic Resonance Imaging. Although commonly used for diagnostic imaging, small molecule contrast agents may suffer from certain disadvantages such as leakage from blood vessel walls leading to short blood circulation time, lower sensitivity, high viscosity, and high osmolality. These compounds generally have been associated with renal complications in some patient populations. This class of small molecule agents is known to clear from the body rapidly, limiting the time over which they can be used to effectively image the vascular system as well as, in regards to other indications, making it difficult to target these agents to disease sites. Thus there is a need for a new class of contrast agents.

Nanoparticles are being widely studied for medical applications, both diagnostic and therapeutic. While only a few nanoparticle-based agents have been clinically approved for magnetic resonance imaging applications and for drug delivery applications, hundreds of such agents are still in development. There is substantial evidence that nanoparticles may provide benefits in efficacy for diagnostics and therapeutics over currently used small molecule-based agents. However, the effects of particle size, structure, and surface properties on the in-vivo bio-distribution and clearance of nanoparticle agents are not well understood. Nanoparticles, depending on their size, tend to stay in the body for longer periods compared to small molecules. In the case of contrast agents, it is preferred to have maximum renal clearance of the agents from the body without causing short term or long term toxicity to any organs.

In view of the above, there is a need for nanoparticle-based contrast agents or imaging agents with improved properties, particularly related to renal clearance and toxicity effects.

### BRIEF DESCRIPTION OF THE INVENTION

The present invention provides a new class of nanoparticle-based contrast agents for X-ray, CT and MRI. The present inventors have found that nanoparticles functionalized with both net positively charged groups and net negatively charged groups surprisingly have improved imaging characteristics compared to small molecule contrast agents. The nanoparticles of the present invention have characteristics that result in reduced retention of the particles in the body compared to other nanoparticles. These nanoparticles may provide improved performance and benefit in one or more of the following areas: robust synthesis, reduced cost, image contrast enhancement, increased blood half life, and decreased toxicity.

The present invention is directed to a composition comprising a nanoparticle, its method of making and method of use.

One aspect of the invention relates to a composition comprising a nanoparticle. The nanoparticle comprises a core and a shell. The shell comprises a plurality of silane moieties. At least one silane moiety of the plurality of silane moieties is functionalized with a net positively charged group and at least one silane moiety of the plurality of silane moieties is functionalized with a net negatively charged group. The net positively charged group and the net negatively charged group reside on different silane moieties. In one embodiment, the at least one silane moiety is functionalized with one positively charged group and the at least one silane moiety is functionalized with one negatively charged group. In one embodiment, the core comprises a transition metal. In another embodiment, the core comprises a derivative of a transition metal selected from the group consisting of oxides, carbides, sulfides, nitrides, phosphides, borides, halides, selenides, tellurides, and combinations thereof. In one embodiment, the core comprises a metal with an atomic number ≧34. For molecular compounds or mixtures of different atoms the atomic number of the compound or the mixture may be represented by ‘effective atomic number,’ Z_{eff}. The Z_{eff} may be calculated as a function of the atomic number of the constituent elements. In such embodiments the core comprises material having an effective atomic number greater than or equal to 34.

In some embodiments, the composition comprises a nanoparticle comprising a tantalum oxide core and a shell. The shell comprises a plurality of silane moieties. The plurality of silane moieties comprises at least one silane moiety functionalized with a net positively charged group and at least one silane moiety functionalized with a net negatively charged group. The net positively charged group and the net negatively charged group reside on different silane moieties. In one embodiment, the ratio of the silane moieties functionalized with the net positively charged groups to the silane moieties functionalized with the net negatively charged groups is in the range from about 0.25 to about 1.75. In one embodiment, the at least one silane moiety is functionalized with one positively charged group and the at least one silane moiety is functionalized with one negatively charged group. In some embodiments, the ratio of the silane moieties functionalized with the one positively charged group to the silane moieties functionalized with the one negatively charged group is about 1. In one embodiment, the nanoparticle has an average particle size up to about 6 nm.

In some other embodiments, the composition comprises a nanoparticle comprising a superparamagnetic iron oxide core and a shell. The shell comprises a plurality of silane moieties. The plurality of silane moieties comprises at least one silane moiety functionalized with a net positively charged group and at least one silane moiety functionalized with a net negatively charged group. The net positively charged group and the net negatively charged group reside on different silane moieties. In one embodiment, the ratio of the silane moieties functionalized with the net positively charged groups to the silane moieties functionalized with the net negatively charged groups is in the range from about 0.25 to about 1.75. In one embodiment, the at least one silane moiety is functionalized with one positively charged group and the at least one silane moiety is functionalized with one negatively charged group. In some embodiments, the ratio of the silane moieties functionalized with the one positively charged group to the silane moieties functionalized with the one negatively charged group is about 1. In another embodiment, the nanoparticle has an average particle size up to about 50 nm.

In one or more embodiments, the invention relates to a diagnostic agent composition. The composition comprises a plurality of nanoparticles, wherein at least one nanoparticle of the plurality of nanoparticles comprises a core and a shell. The shell comprises a plurality of silane moieties. At least one silane moiety of the plurality is functionalized with a net positively charged group and at least one silane moiety of the plurality is functionalized with a net negatively charged group. The net positively charged group and the net negatively charged group reside on different silane moieties. In one embodiment, the at least one silane moiety is functionalized with one positively charged group and the at least one silane moiety is functionalized with one negatively charged group. In some embodiments, the composition further comprises a pharmaceutically acceptable carrier and optionally one or more excipients.

One aspect of the invention relates to methods for making a plurality of nanoparticles. The method comprises (a) providing a core, and (b) disposing a shell on the core, wherein the shell comprises a plurality of silane moieties. The plurality of silane moieties comprises at least one silane moiety functionalized with a net positively charged group and at least one silane moiety functionalized with a net negatively charged group. The net positively charged group and the net negatively charged group reside on different silane moieties. In one embodiment, the ratio of the silane moieties functionalized with the net positively charged groups to the silane moieties functionalized with the net negatively charged groups is in the range from about 0.25 to about 1.75. In one embodiment, the at least one silane moiety is functionalized with one positively charged group and the at least one silane moiety is functionalized with one negatively charged group. In some embodiments, the ratio of the silane moieties functionalized with the one positively charged group to the silane moieties functionalized with the one negatively charged group is about 1.

In some embodiments, the method comprises administering a diagnostic agent composition to a subject and imaging the subject with a diagnostic device. The diagnostic agent composition comprises a plurality of nanoparticles. At least one nanoparticle of the plurality of the nanoparticles comprises a core and a shell. The shell comprises a plurality of silane moieties. At least one silane moiety of the plurality of silane moieties is functionalized with a net positively charged group and at least one silane moiety of the same plurality of silane moieties is functionalized with a net negatively charged group. The net positively charged group and the net negatively charged group reside on different silane moieties. In one embodiment, the at least one silane moiety is functionalized with one positively charged group and the at least one silane moiety is functionalized with one negatively charged group. In one or more embodiments, the method further comprises monitoring delivery of the diagnostic agent composition to the subject with the diagnostic device and diagnosing the subject. In some embodiments, the diagnostic device employs an imaging method chosen from magnetic resonance imaging, optical imaging, optical coherence tomography, X-ray, computed tomography, positron emission tomography, or combinations thereof.

Another aspect of the invention is directed to a method comprising administering a diagnostic agent composition to a subject and imaging the subject with an X-ray device. The diagnostic agent composition comprises a plurality of nanoparticles, wherein at least one nanoparticle of the plurality of the nanoparticles comprises a core and a shell. The shell comprises a plurality of silane moieties. The plurality of silane moieties comprises at least one silane moiety functionalized with a net positively charged group and at least one silane moiety functionalized with a net negatively charged group. The net positively charged group and the net negatively charged group reside on different silane moieties. In one embodiment, the at least one silane moiety is functionalized with one positively charged group and the at least one silane moiety is functionalized with one negatively charged group. In one or more embodiments, the core comprises tantalum oxide.

Another aspect of the invention is directed to a method comprising administering a diagnostic agent composition to a subject and imaging the subject with a Magnetic Resonance Imaging device. The diagnostic agent composition comprises a plurality of nanoparticles, wherein at least one nanoparticle of the plurality of the nanoparticles comprises a core and a shell. The shell comprises a plurality of silane moieties. The plurality of silane moieties comprises at least one silane moiety functionalized with a net positively charged group and at least one silane moiety functionalized with a net negatively charged group. The net positively charged group and the net negatively charged group reside on different silane moieties. In one embodiment, the at least one silane moiety is functionalized with one positively charged group and the at least one silane moiety is functionalized with one negatively charged group. In one or more embodiments, the core comprises superparamagnetic iron oxide.

### DRAWINGS

These and other features, aspects, and advantages of the present invention will become better understood when the following detailed description is read with reference to the accompanying drawings in which like characters represent like parts throughout the drawings, wherein:

FIG. 1 depicts a cross-sectional view of a nanoparticle comprising a core and a shell, in accordance with some embodiments of the present invention.

FIG. 2 describes precursors to negatively charged groups that may be used to functionalize a silane moiety, in accordance with some embodiments of the present invention.

FIG. 3 describes precursors to positively charged groups that may be used to functionalize a silane moiety, in accordance with some embodiments of the present invention.

FIG. 4 describes an example of a silane moiety functionalized with a net positively charged group and a silane moiety functionalized with a net negatively charged group disposed on the core to produce the shell, in accordance with some embodiments of the present invention.

### DETAILED DESCRIPTION

The following detailed description is exemplary and is not intended to limit the invention or the uses of the invention. Furthermore, there is no intention to be limited by any theory presented in the preceding background of the invention or the following detailed description.

In the following specification and the claims which follow, reference will be made to a number of terms having the following meanings. The singular forms “a”, “an” and “the” include plural referents unless the context clearly dictates otherwise. Approximating language, as used herein throughout the specification and claims, may be applied to modify any quantitative representation that could permissibly vary without resulting in a change in the basic function to which it is related. Accordingly, a value modified by a term such as “about” is not to be limited to the precise value specified. In some instances, the approximating language may correspond to the precision of an instrument for measuring the value. Similarly, “free” may be used in combination with a term, and may include an insubstantial number, or trace amounts, while still being considered free of the modified term. For example, free of solvent or solvent-free, and like terms and phrases, may refer to an instance in which a significant portion, some, or all of the solvent has been removed from a solvated material.

As a preliminary matter, the definition of the term “or” for the purpose of the following discussion and the appended claims is intended to be an inclusive “or.” That is, the term “or” is not intended to differentiate between two mutually exclusive alternatives. Rather, the term “or” when employed as a conjunction between two elements is defined as including one element by itself, the other element itself, and combinations and permutations of the elements. For example, a discussion or recitation employing the terminology “A” or “B” includes: “A” by itself, “B” by itself, and any combination thereof, such as “AB” and/or “BA.”

Throughout the following description, “positively charged” and “negatively charged” refer to properties expected under nominal physiological conditions. For instance, the positively charged and the negatively charged groups may behave differently under different pH conditions. For example, a positively charged group may become substantially neutral at high pH, and a negatively charged group may become substantially neutral at low pH.

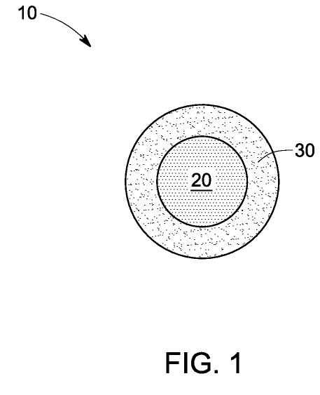

One or more embodiments of the invention are related to a composition comprising a nanoparticle as described in FIG. 1. The nanoparticle 10 composition comprises a core 20, and a shell 30. In one or more embodiments, the core 20 contains a transition metal, for example, a derivative of a transition metal element. The shell 30 comprises a plurality of silane moieties. At least one silane moiety of the plurality is functionalized with a net positively charged group and at least one silane moiety of the same plurality of silane moieties is functionalized with a net negatively charged group. The net positively charged group and the net negatively charged group reside on different silane moieties. In one embodiment, the at least one silane moiety is functionalized with one positively charged group and the at least one silane moiety is functionalized with one negatively charged group.

“Nanoparticle” as used herein refers to particles having a particle size on the nanometer scale, generally less than 1 micrometer. In one embodiment, the nanoparticle has a particle size up to about 50 nm. In another embodiment, the nanoparticle has a particle size up to about 10 nm. In another embodiment, the nanoparticle has a particle size up to about 6 nm.

A plurality of nanoparticles may be characterized by one or more of the following: median particle size, average diameter or particle size, particle size distribution, average particle surface area, particle shape, or particle cross-sectional geometry. Furthermore, a plurality of nanoparticles may have a distribution of particle sizes that may be characterized by both a number-average particle size and a weight-average particle size. The number-average particle size may be represented by S_{N}=Σ(sᵢnᵢ)/Σnᵢ, where nᵢ is the number of particles having a particle size sᵢ. The weight average particle size may be represented by S_{W}=Σ(sᵢnᵢ²)/Σ(sᵢnᵢ). When all particles have the same size, S_{N} and S_{W} may be equal. In one embodiment, there may be a distribution of sizes, and S_{N} may be different from S_{W}. The ratio of the weight average particle size to the number average particle size may be defined as the polydispersity index (S_{PDI}). In one embodiment, SPDI may be equal to about 1. In other embodiments, respectively, S_{PDI} may be in a range of from about 1 to about 1.2, from about 1.2 to about 1.4, from about 1.4 to about 1.6, or from about 1.6 to about 2.0. In one embodiment, S_{PDI} may be in a range that is greater than about 2.0.

In one embodiment, a plurality of nanoparticles may have one of various types of particle size distribution, such as a normal distribution, a monomodal distribution, or a multimodal distribution (for example, a bimodal distribution). Certain particle size distributions may be useful to provide certain benefits. A monomodal distribution may refer to a distribution of particle sizes distributed about a single mode. In another embodiment, populations of particles having two distinct sub-population size ranges (a bimodal distribution) may be included in the composition.

A nanoparticle may have a variety of shapes and cross-sectional geometries that may depend, in part, upon the process used to produce the particles. In one embodiment, a nanoparticle may have a shape that is a sphere, a rod, a tube, a flake, a fiber, a plate, a wire, a cube, or a whisker. A nanoparticle may include particles having two or more of the aforementioned shapes. In one embodiment, a cross-sectional geometry of the particle may be one or more of circular, ellipsoidal, triangular, rectangular, or polygonal. In one embodiment, a nanoparticle may consist essentially of non-spherical particles. For example, such particles may have the form of ellipsoids, which may have all three principal axes of different lengths, or may be oblate or prelate ellipsoids of revolution. Non-spherical nanoparticles alternatively may be laminar in form, wherein laminar refers to particles in which the maximum dimension along one axis is substantially less than the maximum dimension along each of the other two axes. Non-spherical nanoparticles may also have the shape of frusta of pyramids or cones, or of elongated rods. In one embodiment, the nanoparticles may be irregular in shape. In one embodiment, a plurality of nanoparticles may consist essentially of spherical nanoparticles.

A population of nanoparticles may have a high surface-to-volume ratio. A nanoparticle may be crystalline or amorphous. In one embodiment, a single type (size, shape, and the like) of nanoparticle may be used, or mixtures of different types of nanoparticles may be used. If a mixture of nanoparticles is used they may be homogeneously or non-homogeneously distributed in the composition.

In some embodiments, the nanoparticles may not be strongly agglomerated and/or aggregated, with the result that the particles may be relatively easily dispersed in the composition. An aggregate may include more than one nanoparticle in physical contact with one another, while agglomerates may include more than one aggregate in physical contact with one another. In some other embodiments, some of the nanoparticles of the plurality of nanoparticles may form aggregate/agglomerate.

In one embodiment, the core comprises a transition metal. As used herein, “transition metal” refers to elements from groups 3-12 of the Periodic Table. In certain embodiments, the core comprises one or more derivatives of transition metal elements, such as oxides, carbides, sulfides, nitrides, phosphides, borides, halides, selenides, and tellurides, that contain one or more of these transition metal elements. Accordingly, in this description the term “metal” does not necessarily imply that a zero-valent metal is present; instead, the use of this term signifies the presence of a metallic or nonmetallic material that contains a transition metal element as a constituent.

In some embodiments, the nanoparticle may comprise a single core. In some other embodiments, the nanoparticle may comprise a plurality of cores. In embodiments where the nanoparticle comprises plurality of cores, the cores may be the same or different. In some embodiments, the nanoparticle composition comprises at least two cores. In other embodiments, each of the nanoparticle composition comprises only one core.

In some embodiments, the core comprises a derivative of a single transition metal. In another embodiment, the core comprises derivatives of two or more transition metals. In embodiments where the core comprises two or more transition metal derivatives, the transition metal element or the transition metal cation may be of the same element or of two or more different elements. For example, in one embodiment, the core may comprise a single metal derivative, such as tantalum oxide or iron oxide. In another embodiment, the core may comprise derivatives of two or more different metal elements, for example tantalum oxide and hafnium oxide or tantalum oxide and hafnium nitride, or oxides of iron and manganese. In another embodiment, the core may comprise two or more derivatives of the same metal element, for example tantalum oxide and tantalum sulfide.

In one embodiment, the core creates a contrast enhancement in X-ray or computed tomography (CT) imaging. A conventional CT scanner uses a broad spectrum of X-ray energy between about 10 keV and about 150 keV. Those skilled in the art will recognize that the amount of X-ray attenuation passing through a particular material per unit length is expressed as the linear attenuation coefficient. At an X-ray energy spectrum typical in CT imaging, the attenuation of materials is dominated by the photoelectric absorption effect and the Compton Scattering effect. Furthermore, the linear attenuation coefficient is well known to be a function of the energy of the incident X-ray, the density of the material (related to molar concentration), and the atomic number (Z) of the material. For molecular compounds or mixtures of different atoms the ‘effective atomic number,’ Z_{eff}, can be calculated as a function of the atomic number of the constituent elements. The effective atomic number of a compound of known chemical formula is determined from the relationship:

$${Z}_{eff} = {[ {\sum }_{k = 1}^{P} {w}_{{f}_{k}} {Z}_{k}^{β} ]}^{1 / β} \qquad ( Eq . 1 )$$

where Zₖ is the atomic number of metal elements, P is the total quantity of metal elements, and w_{f}_{ₖ} is the weight fraction of metal elements with respect to the total molecular weight of the molecule (related to the molar concentration). The optimal choice of the incident X-ray energy for CT imaging is a function of the size of the object to be imaged and is not expected to vary much from the nominal values. It is also well known that the linear attenuation coefficient of the contrast agent material is linearly dependent on the density of the material, i.e., the linear attenuation coefficient can be increased if the material density is increased or if the molar concentration of the contrast material is increased. However, the practical aspects of injecting contrast agent material into patients, and the associated toxicity effects, limit the molar concentration that can be achieved. Therefore it is reasonable to separate potential contrast agent materials according to their effective atomic number. Based on simulations of the CT contrast enhancement of typical materials for a typical CT energy spectrum with a molar concentration of approximately 50 mM, it is estimated that materials with effective atomic number greater than or equal to 34 may yield appropriate contrast enhancement of about 30 Hounsfield units (HU), or 3% higher contrast than water. Therefore, in certain embodiments the core comprises material having an effective atomic number greater than or equal to 34. See, e.g., Chapter 1 in Handbook of Medical Imaging, Volume 1. Physics and Psychophysics, Eds. J. Beutel, H. L. Kundel, R. L. Van Metter, SPIE Press, 2000.

A core that contains transition metals with relatively high atomic number as described above may provide embodiments having certain desirable characteristics. In such embodiments, the core is substantially radiopaque, meaning that the core material allows significantly less X-ray radiation to pass through than materials typically found in living organisms, thus potentially giving the particles utility as contrast agents in X-ray imaging applications, such as computed tomography (CT). Examples of transition metal elements that may provide this property include tungsten, tantalum, hafnium, zirconium, molybdenum, silver, and zinc. Tantalum oxide is one particular example of a suitable core composition for use in X-ray imaging applications. In one or more embodiments, the core of the nanoparticle comprises tantalum oxide and the nanoparticle has a particle size up to about 6 nm. This embodiment may be particularly attractive for applications in imaging techniques that apply X-rays to generate imaging data, due to the high degree of radiopacity of the tantalum-containing core and the small size that aids rapid renal clearance, for example.

In some embodiments, the core of the nanoparticle comprises a material comprising at least about 30% transition metal element by weight. In certain embodiments, the core of the nanoparticle comprises a material comprising at least about 50% transition metal element by weight. In still further embodiments, the core of the nanoparticle comprises a material comprising at least about 75% transition metal element by weight. Having a high transition metal element content in the core provides the nanoparticle with higher degree of radiopacity per unit volume, thereby imparting more efficient performance as a contrast agent.

In another embodiment, the core comprises material that exhibits magnetic behavior, including, for example, superparamagnetic behavior. The “superparamagnetic material” as used herein refers to material that may exhibit a behavior similar to paramagnetism even when at temperatures below the Curie or the Neel temperature. Examples of potential magnetic or superparamagnetic materials include materials comprising one or more of iron, manganese, copper, cobalt, nickel or zinc. In one embodiment, the superparamagnetic material comprises superparamagnetic iron oxide. In some embodiments, the nanoparticles of the present invention may be used as magnetic resonance (MR) contrast agents. These nanoparticles may yield a T2*, T2, or T1 magnetic resonance signal upon exposure to a magnetic field. In one or more embodiments, the core of the nanoparticle comprises superparamagnetic iron oxide and the nanoparticle has a particle size up to about 50 nm.

In one embodiment, the nanoparticle 10 comprises a shell 30 substantially covering the core 20. This shell 30 may serve to stabilize the core 20, i.e., the shell 30 may prevent one core 20 from contacting an adjacent core 20, thereby preventing a plurality of such nanoparticles 10 from aggregating or agglomerating as described herein, or by preventing leaching of metal or metal oxide, for instance, on the time scale of in-vivo imaging experiments. In one embodiment, the shell 30 may be of a sufficient thickness to stabilize the core 20 and prevent such contact. In one embodiment, the shell 30 has an average thickness up to about 50 nm. In another embodiment, the shell 30 has an average thickness up to about 3 nm.

As used herein, the term “substantially covering” means that a percentage surface coverage of the nanoparticle is greater than about 20%. Percentage surface coverage refers to the ratio of the nanoparticle surface covered by the shell to the surface not covered by the shell. In some embodiments, the percentage surface coverage of the nanoparticle may be greater than about 40%.

In some embodiments, the shell may facilitate improved water solubility, reduce aggregate formation, reduce agglomerate formation, prevent oxidation of nanoparticles, maintain the uniformity of the core-shell entity, or provide biocompatibility for the nanoparticles. In another embodiment, the material or materials comprising the shell may further comprise other materials that are tailored for a particular application, such as, but not limited to, diagnostic applications. For instance, in one embodiment, the nanoparticle may further be functionalized with a targeting ligand. The targeting ligand may be a molecule or a structure that provides targeting of the nanoparticle to a desired organ, tissue or cell. The targeting ligand may include, but is not limited to, proteins, peptides, antibodies, nucleic acids, sugar derivatives, or combinations thereof. In some embodiments, the nanoparticles may further comprise targeting agents such that, when used as contrast agents, the particles can be targeted to specific diseased areas of the subject's body. In some embodiments, the nanoparticles may be used as blood pool agents.

The cores may be covered with one or more shells. In some embodiments, a plurality of cores may be covered with the same shell. In one embodiment, a single shell may cover all the cores present in the nanoparticle composition. In some embodiments, the individual cores may be covered with one or more shells. In another embodiment, all the cores present in the nanoparticle may be covered with two or more shells. In one embodiment, an individual shell may comprise the same material as companion shells. In another embodiment, the shells may comprise different materials. In embodiments where the core is covered with more than one shell, the shells may be of the same or of different material.

In some embodiments, the shell comprises a plurality of silane moieties. The term “plurality of silane moieties” refers to multiple instances of one particular silane moiety, or multiple instances of two or more different silane moieties. In one or more embodiments, at least one silane moiety of the plurality of silane moieties is functionalized with a net positively charged group and at least one silane moiety of the plurality of silane moieties is functionalized with a net negatively charged group. The silane moieties may undergo chemical modifications during the functionalization of the silane moieties with the positively charged group or the negatively charged group or precursors to either group.

In one embodiment, at least one silane moiety of the plurality of silane moieties is functionalized with one positively charged group and at least one other silane moiety of the same plurality of silane moieties is functionalized with one negatively charged group. In such embodiments, the net positively charged group consists of only one positively charged group and the net negatively charged group consists of only one negatively charged group respectively. In some embodiments, the ratio of the silane moieties functionalized with the one positively charged group to the silane moieties functionalized with the one negatively charged group is about 1. In such embodiments, the shell may comprise a near equal number of silane functionalized positively charged groups and silane functionalized negatively charged groups. In such embodiments, the nanoparticle may behave as a neutral particle.

In some other embodiments, at least one silane moiety is functionalized with a net positively charged group and at least one other silane moiety is functionalized with a net negatively charged group. In another embodiment, a plurality of silane moieties is functionalized with net positively charged groups and a plurality of silane moieties is functionalized with net negatively charged groups. In some embodiments, each of the silane moieties of the plurality is functionalized with a net positively charged group or a net negatively charged group. In some embodiments, the ratio of the silane moieties functionalized with the net positively charged groups to the silane moieties functionalized with the net negatively charged groups is in the range from about 0.25 to about 1.75. In some other embodiments, the ratio of the silane moieties functionalized with the net positively charged groups to the silane moieties functionalized with the net negatively charged groups is about 1. In such embodiments, the shell may comprise a near equal number of silane functionalized net positively charged groups and silane functionalized net negatively charged groups. In such embodiments, the nanoparticle may behave as a neutral particle.

In one or more embodiments, the plurality of silane moieties may comprise another type of silane functionalized group in addition to the silane functionalized net positively and net negatively charged groups. In one embodiment, at least one of the silane moieties of the plurality of silane moieties is functionalized with a neutral group; one example of such a neutral group is an alkyl group, although those skilled in the art will recognize that there are many possible neutral groups. In such embodiments, the shell comprises a mixture of at least one silane functionalized net positively charged group, at least one silane functionalized net negatively charged group, and at least one silane functionalized neutral group. In some embodiments, the ratio of silane functionalized charged groups to the silane functionalized neutral groups is in the range from about 0.01 to about 100. In such embodiments, the shell may comprise a plurality of silane functionalized positively charged groups, a plurality of silane functionalized negatively charged groups and two or more silane functionalized neutral groups. The silane functionalized net positively charged groups and the silane functionalized net negatively charged groups, in combination, form the silane functionalized charged groups. In some other embodiments, the ratio of the silane functionalized charged groups to the silane functionalized neutral groups is in the range from about 0.1 to about 20.

In one embodiment, all the silane moieties may be the same type, i.e., only one single type of silane moiety, all the net positively charged groups may be the same type, i.e., only one single type of net positively charged group, and also all the net negatively charged groups may be the same type, i.e., only one single type of net negatively charged group. In another embodiment, the silane moieties may be the same but all the net positively charged groups or all the net negatively charged groups may not be the same. For example, the shell may comprise two or more different types of silane functionalized net positively charged groups and two or more different types of silane functionalized net negatively charged groups. In one embodiment, the shell may comprise one type of silane moiety functionalized with a net positively charged group of a first type and the same type of silane moiety functionalized with a net negatively charged group of a second type. In another embodiment, the shell may comprise a plurality of one type of silane moiety functionalized with a first type of positively charged groups, and a plurality of the same type of silane moiety functionalized with two or more different types of negatively charged groups, i.e., some of the silane moieties may be functionalized with a second type of negatively charged groups and some of the silane moieties may be functionalized with a third type of negatively charged groups.

As used herein, the term “net positively charged group” refers to a single positively charged group, a plurality of positively charged groups, or combination of plurality of positively and negatively charged groups, such that the net charge of the combination is positive. In some embodiments, the net positively charged group refers to a single positively charged group, or one positively charged group, such as a protonated primary amine or a quarternary alkyl amine. In some embodiments, the single or the plurality of positively charged groups may further contain one or more neutral groups, such as an alkyl or an aryl group. In some embodiments, the net positively charged group may comprise of plurality of positively charged groups. In such embodiments, the positively charged groups may or may not be the same. For example, in some embodiments, the net positively charged group comprises a plurality of protonated pyrimidines and a plurality of protonated secondary amines. In some other embodiments, the net positively charged group comprises protonated pyrimidines, protonated secondary amines and quarternary amines. In some embodiments, the net positively charged group may refer to a combination of plurality of positively charged groups, plurality of negatively charged groups, and optionally one or more neutral groups. In such embodiments, the plurality of positively and negatively charged groups is present in a ratio such that the net charge of the combination is positive. In embodiments where the net positively charged group comprises a plurality of positively charged groups, a plurality of negatively charged groups and a plurality of neutral groups, the positively or negatively charged groups or the neutral groups may be the same or different. For example the net positively charged group may comprise a plurality of protonated imidazoles, a plurality of protonated primary amines, a plurality of deprotonated carboxylic acids, a plurality of deprotonated sulfonic acids and a plurality of alkyl derivatives, provided that the net charge of the combination is positive.

Similarly, the “net negatively charged group” refers to a single negatively charged group, a plurality of negatively charged groups, or a combination of plurality of positively and negatively charged groups, in a ratio such that the net charge of the combination is negative. In some embodiments, the net negatively charged group refers to a single negatively charged group, or one negatively charged group, such as a deprotonated carboxylic acid or a deprotonated sulfinic acid. In some embodiments, the single or the plurality of negatively charged groups may further contain one or more neutral groups, such as an alkyl or an aryl group. In some other embodiments, the net negatively charged group may comprise a plurality of negatively charged groups. In such embodiments, the negatively charged groups may or may not be the same. For example, in one embodiment, the net negatively charged group may comprise deprotonated sulfonic acids, deprotonated phosphonic acids and deprotonated carboxylic acids. In some other embodiments, the net negatively charged group comprises a plurality of deprotonated sulfonic acids or a plurality of deprotonated phosphonic acids. In some embodiments, the net negatively charged group may refer to a combination of a plurality of positively charged groups, a plurality of negatively charged groups, and optionally one or more neutral groups. In embodiments where the net negatively charged group comprises a plurality of positively charged groups, a plurality of negatively charged groups and a plurality of neutral groups, the positively or negatively charged groups or the neutral groups may be the same or different. For example the net negatively charged group may comprise a plurality of protonated imidazoles, a plurality of protonated primary amines, a plurality of deprotonated carboxylic acids, a plurality of deprotonated sulfonic acids and a plurality of alkyl derivatives, provided that the net charge of the combination is negative.

Examples of suitable net positively charged groups include, without limitation, protonated primary amines, protonated secondary amines, protonated tertiary alkyl amines, protonated amidines, protonated guanidines, protonated pyridines, protonated pyrimidines, protonated pyrazines, protonated purines, protonated imidazoles, protonated pyrroles, quaternary alkyl amines, quaternary imidazoles, and combinations thereof. Examples of suitable net negatively charged groups include, without limitation, deprotonated carboxylic acids, deprotonated sulfonic acids, deprotonated sulfinic acids, deprotonated phosphonic acids, deprotonated phosphoric acids, deprotonated phosphinic acids, and combinations thereof.

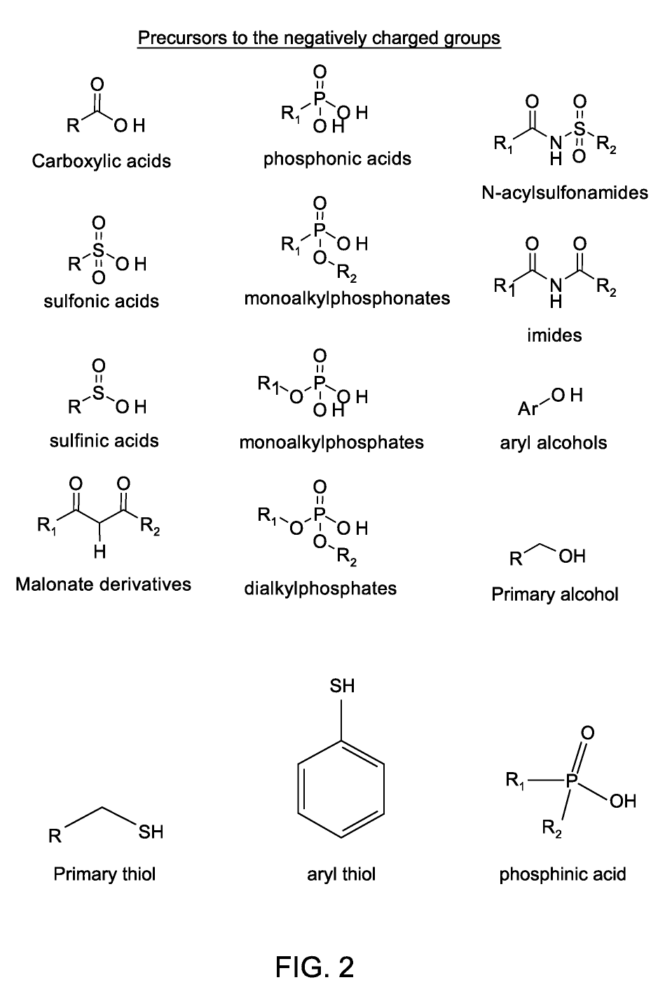

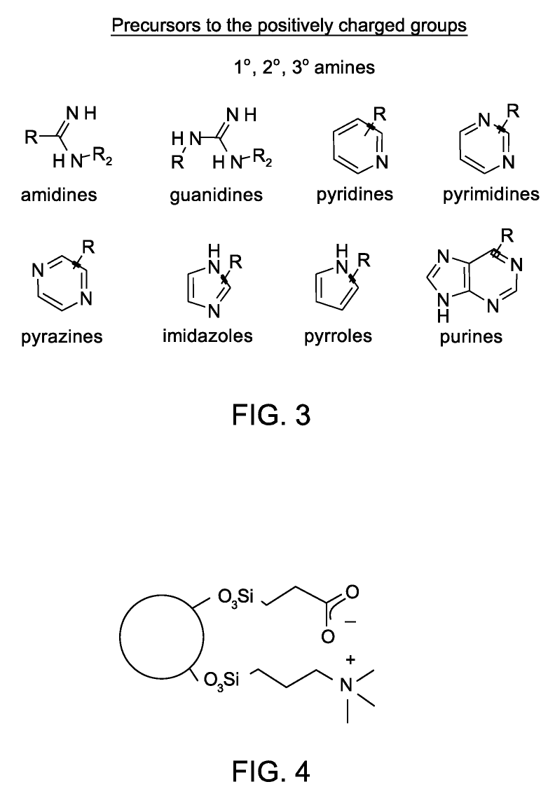

In some embodiments, the “net positively charged group” or the “net negatively charged group” refers to a precursor to a positively charged group or a negatively charged group. In such embodiments, the precursor undergoes a secondary or subsequent chemical reaction to form a positively charged or negatively charged group. Examples of such precursors are illustrated in FIGS. 2 and 3.

In some embodiments, the at least one silane moiety of the plurality of silane moieties is connected to the net positively charged group or to the net negatively charged group via a spacer group. In such embodiments, a silicon atom of the silane moiety is connected to the positively or negatively charged group via the spacer group. In another embodiment, each of the silane moieties is connected to the net positively charged group and to the net negatively charged group via a spacer group. The spacer groups may be same or different. In one or more embodiments, the spacer group is selected from the group consisting of alkyl groups, aryl groups, substituted alkyl and aryl groups, heteroalkyl groups, heteroaryl groups, ethers, amides, esters, carbamates, ureas, straight chain alkyl groups of 1 to 10 carbon atoms in length, and combinations thereof.

In some embodiments, the silane moiety, or the silane functionalized net positively or net negatively charged group may be derived from a hydrolysis product of a precursor trialkoxy silane. In some embodiments, the precursor trialkoxy silane is selected from the group consisting of (N,N-dimethylaminopropyl) trimethoxysilane, 3-N-methylaminopropyl trimethoxysilane, 3-aminopropyltrimethoxysilane, N-trimethoxysilylpropyl-N,N,N-trimethylammonium chloride, 3-(4,5-dihydroimidazol-1-yl) propyltriethoxysilane, and combinations thereof. In another embodiment, the precursor trialkoxy silane is selected from the group consisting of 2-(carbomethoxy)ethyltrimethoxysilane, acetoxypropyltrimethoxysilane, 3-mercaptopropyltrimethoxysilane, and combinations thereof.

Another aspect of the invention relates to a diagnostic agent composition. The diagnostic agent composition comprises a plurality of the nanoparticles 10 described previously. In one embodiment, the diagnostic agent composition further comprises a pharmaceutically acceptable carrier and optionally one or more excipients. In one embodiment, the pharmaceutically acceptable carrier may be substantially water. Optional excipients may comprise, for example one or more of salts, disintegrators, binders, fillers, or lubricants.

In one embodiment, the plurality of nanoparticles may have a median particle size up to about 50 nm. In another embodiment, the plurality of nanoparticles may have a median particle size up to about 10 nm. In another embodiment, the plurality of nanoparticles may have a median particle size up to about 6 nm. A small particle size may be advantageous in, for example, facilitating clearance from kidneys and other organs.

One aspect of the invention relates to methods for making a plurality of nanoparticles. In general, one method comprises (a) providing a core, and (b) disposing a shell on the core, wherein the shell comprises a plurality of silane moieties. At least one silane moiety of the plurality of silane moieties is functionalized with a net positively charged group and at least one silane moiety of the plurality of silane moieties is functionalized with a net negatively charged group. The net positively charged group and the net negatively charged group reside on different silane moieties. In one embodiment, the at least one silane moiety is functionalized with one positively charged group and the at least one silane moiety is functionalized with one negatively charged group.

In one or more embodiments, the step of providing a core comprises providing a first precursor material, wherein the first precursor material comprises at least one transition metal. In one embodiment, the first precursor material may react with an organic acid to generate the core comprising at least one transition metal. The term “reacts” includes mixing the reactants and allowing them to interact. In one embodiment, the first precursor material may decompose to generate the core. In another embodiment, the first precursor material may hydrolyze to generate the core. In one embodiment, the core may comprise metal oxide. The metal oxide core may be synthesized upon the hydrolysis of a metal alkoxide in the presence of an organic acid. The metal alkoxide may be a tantalum alkoxide such as tantalum pentaethoxide. The organic acid may be, for instance, a carboxylic acid such as isobutyric acid. The hydrolysis reaction may be carried out in the presence of an alcohol solvent, such as 1-propanol or methanol. Nanoparticle synthesis methods are well known in the art and any suitable method for making a nanoparticle core of an appropriate material may be suitable for use in this method.

In one or more embodiments, the step of disposing a shell comprises providing a second precursor material. In one or more embodiments the second precursor material comprises a silane moiety or a precursor to a silane moiety. In one or more embodiments, the second precursor material comprises a trialkoxy silane or a hydrolysis product of a trialkoxy silane. In one embodiment, the silane moiety comprises at least one alkoxy group. The silane moiety may react with the core to form a shell comprising a silane moiety. In one or more embodiments, the silane moiety is mixed with the core and allowed to react. In some embodiments, the precursor to the silane moiety may undergo a hydrolysis reaction in the presence of the core. In some embodiments, a net positively charged group is allowed to react with the silane moiety to form a silane functionalized net positively charged group. During the reaction of the net positively charged group with the silane moiety, both the silane moiety and the net positively charged group may undergo chemical modifications. In one or more embodiments, a net negatively charged group may be allowed to react with the silane moiety to form a silane functionalized net negatively charged group. During the reaction of the net negatively charged group with the silane moiety, both the silane moiety and the net negatively charged group may undergo chemical modifications.

In one or more embodiments, the second precursor material comprises a silane functionalized net positively charged group, a silane functionalized net negatively charged group, or a silane functionalized with a precursor to a net positively or net negatively charged group. In some embodiments, the silane functionalized net positively charged group or silane functionalized net negatively charged group may undergo a hydrolysis reaction in the presence of the core.

In some embodiments, the silane moiety may be functionalized with at least one net positively or net negatively charged group or at least one precursor to a net positively or net negatively charged group. In embodiments wherein the silane moiety is functionalized with a precursor to a net positively or negatively charged group, the silane moiety, disposed onto the core, may not be charged in nature, but may subsequently react with an appropriate reagent to convert the precursor into a net positively or net negatively charged group. In one or more embodiments, the second precursor material comprises the silane functionalized net positively or net negatively charged group or precursor to a silane functionalized net positively or negatively charged group, such as one or more of the precursor trialkoxy silanes described above.

In embodiments wherein the silane moiety of the second precursor material is functionalized with at least one precursor to a net positively charged group, the precursor may undergo a chemical reaction/conversion to form the net positively charged group. In such embodiments, the converting step may take place after the silane moiety of the second precursor material has been disposed on the core. In some embodiments, the converting step may take place in-situ. The converting step may comprise protonation or alkylation of the functionalized silane moiety of the second precursor material in the presence of the core. Similarly, in embodiments wherein the silane moiety of the second precursor material is functionalized with at least one precursor to a net negatively charged group, the precursor may undergo a chemical reaction/conversion to form the net negatively charged group. In such embodiments, the converting step may take place after the silane moiety of the second precursor material has been disposed on the core. In some embodiments, the converting step may take place in-situ. The converting step may comprise hydrolysis or oxidation of the functionalized silane moiety of the second precursor material in the presence of the core.

It will be understood that the order and/or combination of steps may be varied. Thus, according to some embodiments, steps (a) and (b) occur as sequential steps so as to form the nanoparticle from the core and the second precursor material. By way of example and not limitation, in some embodiments, the first precursor material comprises at least one transition metal; wherein the core comprises an oxide of the at least one transition metal; and step (a) further comprises hydrolysis of the first precursor material. According to some embodiments, the first precursor material is an alkoxide or halide of the transition metal, and the hydrolysis process includes combining the first precursor material with an acid and water in an alcoholic solvent. In some embodiments, the silane may comprise polymerizable groups. The polymerization may proceed via acid catalyzed condensation polymerization. In some other embodiments, the silane moiety may be physically adsorbed on the core. In some embodiments, the silane moiety may be further functionalized with other polymers. The polymer may be water soluble and biocompatible. In one embodiment, the polymers include, but are not limited to, polyethylene glycol (PEG), polyethylene imine (PEI), polymethacrylate, polyvinylsulfate, polyvinylpyrrolidinone, or combinations thereof.

In another embodiment, the core and the second precursor material may be brought into contact with each other. In one embodiment, the second precursor material may comprise a silicon containing species such as an organofunctional trialkoxysilane or mixture of organofunctional trialkoxysilanes. At least one of the organofunctional trialkoxy silanes may contain at least one net positively charged group or at least one net negatively charged group or a precursor to a net positively or negatively charged group, such that each nanoparticle, on average, may contain at least one net positively charged group and at least one net negatively charged group or precursor to a net positively or net negatively charged group. In one embodiment, each nanoparticle may contain, on average, a plurality of silane functionalized net positively charged groups and a plurality of silane functionalized net negatively charged groups or precursors to silane functionalized net positively or net negatively charged groups. In other embodiments, the core may be treated with a mixture containing at least two silane moieties. In one embodiment, one silane moiety is functionalized with a net positively group or a precursor to a net positively charged group and the second silane moiety is functionalized with a net negatively charged group, or a precursor to a net negatively charged group. In another embodiment, one silane moiety is functionalized with a net positively or net negatively charged group, or a precursor to a net positively or net negatively charged group, and the second silane moiety may not be functionalized with any net positively or net negatively charged group, but rather be functionalized with a net neutral group. The charged silane moieties may be added simultaneously or sequentially. In some embodiments, one or more silane functionalized net positively or net negatively charged groups, or a precursor to a silane functionalized net positively or net negatively charged group, may be added to a reaction mixture comprising the cores, non-functionalized silane moieties or silane moieties functionalized with neutral groups, either simultaneously or sequentially.

In one embodiment, a tantalum oxide core may be allowed to react with a mixture of silanes such as carbomethoxyethyltrimethoxysilane (CMETS) and dimethylaminopropyl-trimethoxysilane, to produce a nanoparticle comprising a tantalum oxide core and a shell comprising at least one silane functionalized net positively charged group and a precursor to at least one silane functionalized net negatively charged group. This precursor may later convert to a negatively charged group upon exposure to a suitable environment, the characteristics of which environment would be understood to one skilled in the art based on the identity of the precursor and its related chemical properties. This conversion may be effected in-situ or after isolating the particles from the reaction medium.

In one embodiment, the method further comprises fractionating the plurality of nanoparticles. The fractionating step may include filtering the plurality of nanoparticles. In another embodiment, the method may further comprise purifying the plurality of nanoparticles. The purification step may include use of dialysis, tangential flow filtration, diafiltration, or combinations thereof. In another embodiment, the method further comprises isolation of the purified nanoparticles.

In combination with any of the above-described embodiments, some embodiments relate to a method for making a diagnostic agent composition for X-ray/computed tomography or MRI. The diagnostic agent composition comprises a plurality of nanoparticles. In some embodiments, the median particle size of the plurality of nanoparticles may not be more than about 10 nm, for example not more than about 7 nm, and in particular embodiments not more than about 6 nm. It will be understood that according to some embodiments, the particle size of the plurality of nanoparticles may be selected so as to render the nanoparticle substantially clearable by a mammalian kidney, such as a human kidney.

In some embodiments, the present invention is directed to a method of use of the diagnostic agent composition comprising a plurality of the nanoparticles described herein. In some embodiments, the method comprises the in-vivo or in-vitro administration of the diagnostic agent composition to a subject, which in some instances may be a live subject, such as a mammal, and subsequent image generation of the subject with an X-ray/CT, or MRI device. The nanoparticles, as described above, comprise a core and a shell, wherein the shell comprises at least one silane functionalized net positively charged group and at least one silane functionalized net negatively charged group. The net positively charged group and the net negatively charged group reside on different silane moieties. In one embodiment, the at least one silane moiety is functionalized with one positively charged group and the at least one silane moiety is functionalized with one negatively charged group. In one embodiment, the core comprises tantalum oxide. In another embodiment, the core comprises superparamagnetic iron oxide. The nanoparticle may be introduced to the subject by a variety of known methods. Non-limiting examples for introducing the nanoparticle to the subject include intravenous, intra-arterial or oral administration, dermal application, or direct injection into muscle, skin, the peritoneal cavity or other tissues or bodily compartments.

In another embodiment, the method comprises administering the diagnostic agent composition to a subject, and imaging the subject with a diagnostic device. The diagnostic device employs an imaging method, examples of which includes, but is not limited to, MRI, optical imaging, optical coherence tomography, X-ray, computed tomography, positron emission tomography, or combinations thereof. The diagnostic agent composition, as described above, comprises a plurality of the nanoparticles 10.

In one embodiment, the methods described above for use of the diagnostic contrast agent further comprise monitoring delivery of the diagnostic agent composition to the subject with the diagnostic device, and diagnosing the subject; in this method data may be compiled and analyzed generally in keeping with common operation of medical diagnostic imaging equipment. The diagnostic agent composition may be an X-ray or CT contrast agent, for example, such as a composition comprising a tantalum oxide core. The diagnosing agent composition may provide for a CT signal in a range from about 100 Hounsfield to about 5000 Hounsfield units. In another example, the diagnostic agent composition may be a MRI contrast agent, such as an agent comprising a superparamagnetic iron oxide core.

One embodiment of the invention provides a method for determination of the extent to which the nanoparticles 10 described herein, such as nanoparticles having tantalum oxide or iron oxide cores, are distributed within a subject. The subject may be a mammal or a biological material comprising a tissue sample or a cell. The method may be an in-vivo or in-vitro method. The nanoparticle may be introduced to the subject by a variety of known methods. Non-limiting examples for introducing the nanoparticle to the subject include any of the known methods described above. In one embodiment, the method comprises (a) introducing the nanoparticles into the subject, and (b) determining the distribution of the nanoparticles in the subject. Distribution of the nanoparticles within a subject may be determined using a diagnostic imaging technique such as those mentioned previously. Alternatively, the distribution of the nanoparticle in the biological material may be determined by elemental analysis. In one embodiment, Inductively Coupled Plasma Mass Spectroscopy (ICP-MS) may be used to determine the concentration/amount of the nanoparticle components in the biological material.

The following examples are included to demonstrate particular embodiments of the present invention. It should be appreciated by those of skill in the art that the methods disclosed in the examples that follow merely represent exemplary embodiments of the present invention. However, those of skill in the art should, in light of the present disclosure, appreciate that many changes can be made in the specific embodiments described and still obtain a like or similar result without departing from the spirit and scope of the present invention.

### EXAMPLES

Practice of the invention will be still more fully understood from the following examples, which are presented herein for illustration only and should not be construed as limiting the invention in any way.

The abbreviations used in the example section are expanded as follows: “mg”: milligrams; “mL”: milliliters; “mg/mL”: milligrams per milliliter; “mmol”: millimoles; “μL” and “μLs”: microliters; “LC”: Liquid Chromatography; “DLS”: Dynamic Light Scattering; “DI”: deionized water; “ICP”: Inductively Coupled Plasma.

Unless otherwise noted, all reagent-grade chemicals were used as received, and Milli-Q water was used in the preparation of all aqueous solutions.

### Synthesis of Tantalum Oxide Nanoparticles and Reaction with 2-(carbomethoxy)ethyltrimethoxysilane and 3-(trimethoxysilyl)propyl-N,N,N-trimethylammonium Chloride to Form Shells on the Tantalum Oxide Nanoparticle Cores

To 680 mL anhydrous methanol (Aldrich SureSeal) in a 2 L flask was added 10 mL isobutyric acid and 2.78 mL deuterium oxide at room temperature under nitrogen in a glovebox. This mixture was stirred for 40 minutes after which tantalum ethoxide (37.36 g) was added in a drop-wise manner. The addition took on the order of 15-20 minutes. The hydrolysis reaction was allowed to stir for 5 hours, after which the flask was removed from the glove box and rendered inert using a Schlenck-/vacuum-line manifold. A mixture of trimethoxysilanes that included 2-(carbomethoxy)ethyltrimethoxysilane (19.16 g) combined with 3-(trimethoxysilyl)propyl-N,N,N-trimethylammonium chloride (47.44 g, it is sold as a ˜50% solution in methanol) was then added directly to the 2 L reaction vessel as quickly as possible. The mixture was refluxed overnight under nitrogen. The next day, the reaction mixture was cooled to room temperature with continued stirring and 6 mL of 0.15 M ammonium hydroxide was added drop-wise. Three hours later, 60 mL Milli-Q water was added drop-wise and the reaction mixture was set to stir overnight at room temperature. Next, 360 mL of 0.67 M HCl was added drop-wise under stirring and the reaction was heated to 50° C. for 5.5-6 hours (pH 1-2). Upon cooling, the reaction was neutralized using 5.92 M ammonium hydroxide to achieve a pH between 7.5-8. To hydrolyze the methyl ester to a carboxylic acid group, all volatiles were removed by rotary evaporation at 50° C. and residual solids were treated with 250 mL of 5 M ammonium hydroxide solution for 3 days while stirring contents in the same 2 L vessel (capped) at room temperature. The hydrolysis reaction was then brought to pH 8 using 3 M HCl to neutralize all hydroxide. Purification of the batch involved filtration through a 0.45 micron filter, followed by fractionation using a tangential flow filtration (TFF) method. To fractionate, the batch was sent through a 50 kDa membrane with the resulting permeate subjected to a 5 kDa dia-filtration. The 50 kDa filtration was carried out using a 0.1 m² 50 kDa molecular-weight cut-off membrane made of polyethersulfone (PES). The batch from the flask was added to the TFF reservoir and the flask was rinsed twice with 200 mL 0.5 M NaCl, adding each wash to the reservoir. After continuously adding/feeding 16 L of 0.5M NaCl to the reservoir, and collecting all permeate, the batch was concentrated to about 1.5 L and then additionally washed with 2 L water. Next, the entire volume of 50 kDa-permeate collected was dia-filtered against a 5 kDa regenerated cellulose (RC) membrane (0.3 m²). Product was concentrated in the reservoir and 18 L of water was used to wash the retenate. The final product, schematically illustrated in FIG. 4, was a nanoparticle of nominally 5 nanometer size, having a tantalum oxide core and a silane shell; the shell comprised nominally equal quantities of silane moieties functionalized with quaternary amine and silane moieties functionalized with carboxylic acid.

Characterization: DLS: Z(eff) 4.8 nm; Si/Ta mol ratio: 1.52 (ICP: 32.5 mg Ta/g and 7.75 mg Si/g); Yield (based on Ta weight): 78%; 1H NMR (ppm): 0.62 (methylene from trimethylammonium silane), 0.83 (methylene from carboxyethyl silane), 1.89 (methylene from trimethylammonium silane), 2.23-2.35 (broad peak from carboxyethyl silane), 3.09 (N-methyl groups of trimethylammonium silane), 3.3 (methylene from trimethylammonium silane).

### Synthesis of Iron Oxide Nanoparticles and Reaction with 2-(carbomethoxy)ethyltrimethoxysilane and dimethylaminopropyl-trimethoxysilane to Form Shells on the Iron Oxide Nanoparticle Core

A 100 mL three-neck flask was charged with 10 mL of anhydrous benzyl alcohol and 353 mg (1 mmol) of Fe(acac)₃, and the mixture was degassed by bubbling N₂ for 5 minutes. The reaction mixture was sealed and heated to 170° C. for 4 hours. The mixture was cooled to room temperature, 75 mL of tetrahydrofuran was added, followed by addition of 521 mg (2.5 eq.) of carbomethoxyethyltrimethoxysilane (CMETS) and 518 mg (2.5 eq.) of dimethylaminopropyl-trimethoxysilane (DMAPS). The mixture was transferred to a pressure vessel and heated at 50° C. for 2 hours, cooled, and 18 mL of isopropyl alcohol and 30 mL of concentrated ammonium hydroxide were added. The mixture was sealed and heated to 50° C. for 16 hours. The mixture was cooled, and the lower aqueous layer was separated and washed twice with 20 mL of hexane. Residual hexane and tetrahydrofuran were removed by rotary evaporation, and the remaining material was dialyzed against water using 10,000 MW regenerated cellulose dialysis tubing, resulting in an aqueous solution of particles with a particle size of 12 nm as measured by dynamic light scattering. The final product was a nanoparticle of nominally 12 nanometer size, having an iron oxide core and a silane shell; the shell comprised nominally equal quantities of silane moieties functionalized with quaternary amine and silane moieties functionalized with carboxylic acid.

### Nanoparticle Biodistribution Studies

In vivo studies were carried out with male Lewis rats with a size range between 150 and 500 grams body weight. Rats were housed in standard housing with food and water ad libitum and a 12 hour day-night lighting cycle. All animals used for biodistribution were otherwise untreated, normal subjects.

Nanoparticles having tantalum oxide cores were administered as a filter-sterilized solution in either water or saline. Administration was performed under isoflurane anesthesia (4% induction, 2% maintenance) via a 26 G catheter inserted into the lateral tail vein. Injection volumes were determined based on the concentration of the nanoparticles in the injectate and the size of the rat, but were generally less than 10% of rodent blood volume. The target dose was 100 mg of core metal (e.g., tantalum) per kg of body weight. Once injected, animals were removed from anesthesia and, after a period of observation for adverse effects, returned to normal housing. At a later period of as short as a few minutes to as long as 6 months, the rats were euthanized, and organs of interest were harvested, weighed, and analyzed for their total metal (e.g., tantalum) content by ICP analysis. Along with the organs, a sample of the injected material was submitted to determine the exact concentration of injectate. These combined data determined the percentage of the injected dose (“% ID”) remained in a tissue of interest. These data were reported either as % ID/organ, or % ID/gram of tissue. Experiments were generally performed with four duplicate rats at each time-point, allowing for the determination of experimental error (±standard deviation).

TABLE 1
Biodistribution of fractionated nanoparticles with non-charged groups
(PHS) and silane functionalized positively and negatively charged
groups (PMZ and mPMZ) in major clearing organs at 1 week
following IV injection.

| | Kidney | Liver | Spleen |
|---|---|---|---|
| Coating | (% ID/organ) | (% ID/organ) | (% ID/organ) |
| diethylphosphatoethyl | 4.20 ± 0.43 | 2.57 ± 0.64 | 0.16 ± 0.05 |
| (PHS) | | | |
| N,N-dimethylaminopropyl | 0.60 ± 0.05 | 0.10 ± 0.01 | ND |
| and 2-carboxyethyl | | | |
| (PMZ) | | | |
| N,N,N- | 0.48 ± 0.06 | 0.13 ± 0.01 | ND |
| trimethylammoniumpropyl | | | |
| and 2-carboxyethyl- | | | |
| (mPMZ) | | | |

The amount of tantalum retained per organ is represented in Table 1 as the fraction of the injected dose. Comparably sized PHS coated nanoparticles are retained at much higher levels (more than one order of magnitude) than either of the PMZ coated nanoparticles tested.

Similarly, when super paramagnetic iron oxide (SPIO) particles are made and coated with either the PHS or PMZ coating, the PMZ coated particles exhibit reduced tissue retention. Such particles were synthesized and administered to rats that were subsequently imaged by magnetic resonance imaging over time post injection. The amount of MR signal observed in the liver due to PMZ-SPIO was substantially less than that observer for PHS-SPIO. This result demonstrates that the particle coatings described herein can be used on different particle cores to achieve the same desired result.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A method comprising:
administering a diagnostic agent composition to a subject; wherein the diagnostic agent composition comprises a plurality of nanoparticles; wherein at least one nanoparticle of the plurality of nanoparticles comprises a core and a shell; wherein
(a) the shell comprises a plurality of silane moieties;
(b) at least one silane moiety is functionalized with a net positively charged group and at least one silane moiety is functionalized with a net negatively charged group; and
(c) the net positively charged group and the net negatively charged group reside on different silane moieties; and
imaging the subject with a diagnostic device.

2. A method comprising:
administering a diagnostic agent composition to a subject; wherein the diagnostic agent composition comprises a plurality of nanoparticles; wherein at least one nanoparticle of the plurality of nanoparticles comprises a core and a shell; and the nanoparticle has a particle size up to about 6 nm; wherein
(a) the core comprises tantalum oxide, and the shell comprises a plurality of silane moieties;
(b) at least one silane moiety is functionalized with a net positively charged group and at least one silane moiety is functionalized with a net negatively charged group; and
(c) the net positively charged group and the net negatively charged group reside on different silane moieties; and
imaging the subject with an X-ray device.

3. A method comprising:
administering a diagnostic agent composition to a subject; wherein the diagnostic agent composition comprises a plurality of nanoparticles; wherein at least one nanoparticle of the plurality of nanoparticles comprises a core and a shell; and the nanoparticle has a particle size up to about 50 nm; wherein
(a) the core comprises superparamagnetic iron oxide, and the shell comprises a plurality of silane moieties;
(b) at least one silane moiety is functionalized with a net positively charged group and at least one silane moiety is functionalized with a net negatively charged group; and
(c) the net positively charged group and the net negatively charged group reside on different silane moieties; and
imaging the subject with a magnetic resonance imaging device.

4. The method of claim 1, wherein a ratio of the silane moieties functionalized with the net positively charged groups to the silane moieties functionalized with the net negatively charged groups is in the range from about 0.25 to about 1.75.

5. The method of claim 4, wherein the ratio of the silane moieties functionalized with the net positively charged groups to the silane moieties functionalized with the net negatively charged groups is about 1.

6. The method of claim 1, wherein the at least one silane moiety is functionalized with one positively charged group, and the at least one silane moiety is functionalized with one negatively charged group.

7. The method of claim 6, wherein a ratio of the silane moieties functionalized with the one positively charged group, to the silane moieties functionalized with the one negatively charged group is about 1.

8. The method of claim 1, wherein the subject is a live subject.

9. The method of claim 1, further comprises monitoring delivery of the diagnostic agent composition to the subject with the diagnostic device; and diagnosing the subject.

10. The method of claim 1, wherein the diagnostic agent composition is administered to a subject in-vitro.

11. The method of claim 1, wherein the diagnostic agent composition is administered to a subject in-vivo.

12. The method of claim 1, wherein the diagnostic device employs an imaging method selected from the group consisting of MRI, optical imaging, optical coherence tomography, X-ray imaging, X-ray computed tomography, positron emission tomography, and combinations thereof.

13. The method of claim 1, wherein the core comprises a transition metal.

14. The method of claim 1, wherein the core comprises a derivative of a transition metal selected from the group consisting of oxides, carbides, sulfides, nitrides, phosphides, borides, halides, selenides, tellurides, and combinations thereof.

15. The method of claim 1, wherein the core comprises a metal with an atomic number ≧34.

16. The method of claim 15, wherein the core comprises a metal selected from the group consisting of tungsten, tantalum, hafnium, zirconium, molybdenum, silver, and combinations thereof.

17. The method of claim 1, wherein the core comprises tantalum oxide.

18. The method of claim 1, wherein the core comprises a superparamagnetic material.

19. The method of claim 18, wherein the superparamagnetic material comprises a metal selected from the group consisting of iron, manganese, copper, cobalt, nickel, zinc, and combinations thereof.

20. The method of claim 1, wherein the core comprises superparamagnetic iron oxide.

21. The method of claim 1, wherein the at least one silane moiety is connected to the net positively charged group or to the net negatively charged group via a spacer group.

22. The method of claim 1, wherein the at least one silane moiety is connected to the net positively charged group via a spacer group.

23. The method of claim 1, wherein the at least one silane moiety is connected to the net negatively charged group via a spacer group.

24. The method of claim 1, wherein the net positively charged group is selected from the group consisting of protonated primary amines, protonated secondary amines, protonated tertiary alkyl amines, protonated amidines, protonated guanidines, protonated pyridines, protonated pyrimidines, protonated pyrazines, protonated purines, protonated imidazoles, protonated pyrroles, quaternary alkyl amines, quaternary imidazoles, and combinations thereof.

25. The method of claim 1, wherein the net negatively charged group is selected from the group consisting of deprotonated carboxylic acids, deprotonated sulfonic acids, deprotonated sulfinic acids, deprotonated phosphonic acids, deprotonated phosphoric acids, deprotonated phosphinic acids, and combinations thereof.

26. The method of claim 21, wherein the spacer group is selected from the group consisting of alkyl groups, aryl groups, substituted alkyl and aryl groups, heteroalkyl groups, heteroaryl groups, ethers, amides, esters, carbamates, ureas, straight chain alkyl groups of 1 to 10 carbon atoms in length, and combinations thereof.

27. The method of claim 1, wherein the at least one silane moiety comprises a hydrolysis product of a precursor trialkoxy silane.

28. The method of claim 27, wherein the precursor trialkoxy silane is selected from the group consisting of (N,N-dimethylaminopropyl) trimethoxysilane, 3-N-methylaminopropyl trimethoxysilane, 3-aminopropyltrimethoxysilane, N-trimethoxysilylpropyl-N,N,N-trimethylammonium chloride, 3-(4,5-dihydroimidazol-1-yl) propyltriethoxysilane, and combinations thereof.

29. The method of claim 27, wherein the precursor trialkoxy silane is selected from the group consisting of 2-(carbomethoxy)ethyltrimethoxysilane, acetoxypropyltrimethoxysilane, 3-mercaptopropyltrimethoxysilane, and combinations thereof.

30. The method of claim 1, wherein the nanoparticle has a particle size up to about 50 nm.

31. The method of claim 1, wherein the nanoparticle has a particle size up to about 10 nm.

32. The method of claim 1, wherein the nanoparticle has a particle size up to about 6 nm.

33. The method of claim 1, wherein the core comprises a material comprising at least about 30% transition metal element by weight.

34. The method of claim 1, wherein the core comprises a material comprising at least about 50% transition metal element by weight.

35. The method of claim 1, wherein the shell further comprises at least one silane moiety functionalized with a neutral group.

36. The method of claim 35, wherein a ratio of the silane moieties functionalized with charged groups to the silane moieties functionalized with the neutral groups is in the range from about 0.01 to about 100.

37. The method of claim 36, wherein the ratio of the silane moieties functionalized with the charged groups to the silane moieties functionalized with the neutral groups is in the range from about 0.1 to about 20.
